(19) United States
(12) Patent Application Publication
(10) Pub. No.: US 2026/0108471 A1
(43) Pub. Date: Apr. 23, 2026

(54) METHOD OF COATING DRUG-ION EXCHANGE RESIN COMPLEXES

(71) Applicant: Brillian Pharma Inc., Edison, NJ (US)

(72) Inventor: Nuo Wang, Newtown, PA (US)

(73) Assignee: Brillian Pharma Inc., Edison, NJ (US)

(21) Appl. No.: 18/920,661

(22) Filed: Oct. 18, 2024

(51) Int. Cl. A61K 9/50

(52) U.S. Cl. CPC A61K 9/5089; A61K 9/5026; A61K 9/5042

(57) ABSTRACT

This disclosure provides a method of coating ion exchange resin or ion exchange resin-drug solid particles. The coated micronized or non-micronized ion exchange resin or ion exchange resin-drug solid complex particles as prepared by the disclosed methods are suitable for being used directly or as a component for bringing added features for a pharmaceutical product.

### FIELD OF THE INVENTION

This invention relates generally to methods for coating drug-ion exchange resin complexes.

### BACKGROUND OF THE INVENTION

Pharmaceutical oral dosage forms are medications designed to be taken by mouth. They can be in a solid or liquid form, including tablets, capsules, powders, granules, solutions, suspensions, emulsions, and syrups. During drug development for oral delivery, different dosage forms are selected for targeting specific indications, patients, or age groups. The selection involves various considerations, including the patient's age, medical condition, targeted site of action, pharmacokinetics, ability to swallow or chew, and preferences, as well as the properties and limitations of available dosage forms.

Extended release (ER) dosage forms are preferred dosage forms compared to the immediate-release dosage forms. Just like tablets or capsules, the extended release dosage forms are also highly preferred dosage forms for oral liquid drug products by pediatric patients. Extended release formulations allow patients to take medication less frequently compared to immediate-release formulations. Due to the improved dosing convenience, the extended release formulation improves compliance. With fewer dosing frequency, patients are more likely to adhere to their medication regimen, leading to better treatment outcomes.

Despite many benefits, extended release formulations in liquid or powder for liquid dosage forms remain scarce. Many effective drugs in the marketplace are still without extended release formulations in a liquid dosage form. The challenge lies in producing extended release oral liquid formulations due to its manufacturing processes. The particle size of the ER coated components can be significantly larger than that of the drug substances due to the more complex structure of the particles and the limitation of the coating process. Fluid bed coating is a widely used process in the pharmaceutical industry, as it can be used to produce a variety of different coatings. The typical particle size of fluid bed coated particles can vary broadly depending on the specific application and formulation requirements. In general, the particle size range of fluid bed-coated particles typically falls in the range of 100-1000 microns. Particles in such particle size range usually brings undesired properties to oral liquid formulations, such as grittiness mouthfeel, less uniform drug content, or sedimentation issues.

Coating smaller particles of less than 100 microns in the fluid bed coating process can present several challenges. As the particle size decreases, the surface area to volume ratio increases, making it more difficult to properly suspend the particles in the fluid bed due to static charge. This can lead to poor coating uniformity and inconsistent coating. In the fluid bed process, small particles tend to be granulated instead of being coated as discrete particles. Such a process produces coated particles well above an average particle size of several hundred microns. Smaller drug particles require high coating weight gain to achieve extended release properties. Inadequate coating weight gain will cause the product to fail to produce a desired drug release profile. In addition, small particles can be more sensitive to variations in the process parameters, such as air flow rate, inlet temperature, and spray rate. This can make it more challenging to achieve consistent coating quality and uniformity.

Therefore, there is a need for grittiness-free extended release age-appropriate dosage forms.

### SUMMARY OF THE INVENTION

This disclosure addresses the need discussed above in numerous aspects. In one aspect, this disclosure provides a method of coating ion-exchange resin particles. In some embodiments, the method comprises: (a) dissolving a coating polymer in a solvent to form a first solution; (b) suspending ion-exchange resin particles uniformly in the first solution to obtain a solid-in-oil suspension; (c) adding the solid-in-oil suspension to a second solution that contains a surfactant and is immiscible with the first solution to obtain a solid-in-oil-in-water suspension; (d) mixing the solid-in-oil-in-water suspension using agitation to form a solid-in-oil-in-water emulsion/suspension (S/O/W dispersion); (e) passing the S/O/W dispersion through a mechanism or device that exerts a pressure and/or shear force on the S/O/W dispersion and sending resulting dispersion into a third solution that contains a surfactant to obtain a micronized S/O/W dispersion; (f) removing the solvent from the micronized S/O/W dispersion to deposit and harden the coating polymer on the ion-exchange resin particles to obtain coated ion-exchange resin particles; and (g) collecting and optionally drying the coated ion-exchange resin particles.

In some embodiments, the ion-exchange resin particles comprise drug complexed resin particles. In some embodiments, wherein the ion-exchange resin particles comprise micronized drug complexed resin particles having a 90th percentile particle size (D90) of less than 50 microns. In some embodiments, the ion-exchange resin particles comprise cationic or anionic ion-exchange resins.

In some embodiments, the coating polymer forms a barrier on an ion-exchange resin or drug complexed ion-exchange resin. In some embodiments, the coating polymer is insoluble but permeable to a drug in gastrointestinal tract fluid. In some embodiments, the coating polymer comprises a poly(meth)acrylate polymer, a cellulose derived polymer, a polyvinyl acetate polymer, or a combination thereof. In some embodiments, the coating polymer is an ethyl acrylate/methyl methacrylate/trimethylammonioethyl mathacrylate copolymer, an ethyl acrylate/methyl methacrylate copolymer, a butyl/methyl methacrylate/dimthylaminoethyl methacrylate copolymer, a methacrylic acid/ethyl acrylate copolymer, a methacrylic acid/methyl methacrylate copolymer, or a combination thereof. In some embodiments, a concentration of the coating polymer in the first solution is 1-30% wt.

In some embodiments, the solvent is an organic solvent that dissolves the coating polymer while having limited (or less than 10% wt.) solubility in water. In some embodiments, the solvent comprises methylene chloride, ethyl acetate, chloroform, benzene, 1-butanol, diethyl ether, 1,2-dichloroethane, diethylene glycol, methyl t-butyl either, nitromethane, or a combination thereof.

In some embodiments, a volume ratio of the first solution to the second solution is from 1:2 to 1:1000.

In some embodiments, the second solution is an aqueous solution containing a surfactant. In some embodiments, the surfactant comprises an ionic surfactant, a non-ionic surfactant, a polymeric surfactant, or a combination thereof. In some embodiments, the surfactant comprises polyvinyl alcohol. In some embodiments, a concentration of the surfactant in the second solution is from 0.1% to 10% by volume or by weight.

In some embodiments, the agitation is produced by a high shear mechanical process. In some embodiments, the high shear mechanical process is homogenization. In some embodiments, the agitation is produced by a homogenizer operating at a speed of from 500 to 20,000 rpm.

In some embodiments, the mechanism/device has an opening or orifice through which a liquid dispersion can be forced under a pressure of from 100 to 50,000 psi. In some embodiments, the mechanism or device comprises an in-line homogenizer operating at a speed of from 500 to 20,000 rpm.

In some embodiments, a volume ratio of the second solution to the third solution is from 1:0 to 1:1000.

In some embodiments, the third solution is an aqueous solution containing a surfactant. In some embodiments, the surfactant in the third solution is an ionic surfactant, a non-ionic surfactant, a polymeric surfactant, or a combination thereof. In some embodiments, the surfactant in the third solution comprises polyvinyl alcohol. In some embodiments, a concentration of the surfactant in the second solution is from 0.01% to 5% by volume or by weight.

In some embodiments, the solvent is removed by heating and/or vacuum. In some embodiments, the solvent is removed by heating the third solution to a temperature of from 25 to 90° C.

In some embodiments, the coated ion-exchange resin particles are collected by filtration, centrifuging, or centrifuge filtration.

In some embodiments, the coated ion-exchange resin particles are dried by heating or freeze-drying.

In some embodiments, a weight ratio of the coating polymer to the ion-exchange resin particles is from 1% to 80%.

In some embodiments, the ion-exchange resin particles have a particle size of from 0.5 to 500 microns. In some embodiments, the ion-exchange resin particles have a particle size of from 0.5 to 50 microns. In some embodiments, the ion-exchange resin particles have a particle size of from 0.5 to 30 microns.

In some embodiments, the S/O/W dispersion has a droplet size range of from 1 to 1000 microns. In some embodiments, the micronized S/O/W dispersion has a droplet size range of from 0.5 to 50 microns. In some embodiments, the micronized S/O/W dispersion has a droplet size range of from 0.5 to 30 microns.

In some embodiments, the coated ion-exchange resin particles have a particle size of D90 smaller than 50 microns. In some embodiments, the coated ion-exchange resin particles have a particle size of D90 smaller than 30 microns.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combinations of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### DETAILED DESCRIPTION OF THE INVENTION

Although the complexed drug-ion exchange resin by itself can sometimes enable an extended drug release characteristics for some drugs, for most of the drugs, simple complexion with ion exchange resin would not provide the desired therapeutic duration needed for extended release (ER). Therefore, an extended release coat is applied to the surface of the drug-ion exchange resin particles to produce the sustained-release/extended release effect.

It is challenging to coat the drug-ion exchange resin particles with a small particle size using traditional coating processes, especially when the particle size of the coated drug-ion exchange resin particles is required to be less than 30-50 microns (D90). As used herein, D90 is a particle size distribution metric that indicates the point at which 90% of a sample's volume is contained. It describes the diameter where ninety percent of the distribution has a smaller particle size and ten percent has a larger particle size.

The coating process should be able to coat the resin or resinate particles without producing significant agglomeration, so that the smooth mouthfeel of a drug product comprising such coated resonates can be provided. However, traditional fluid bed coating processes (i.e., top spray, bottom spray, and tangential spray processes) could not readily produce a coated product meeting such particle size requirements. These processes typically produce granulated or aggregated products when fine particles are coated.

### Methods for Coating Drug Ion-Exchange Resin Complexes

Accordingly, this disclosure provides methods for coating ion exchange resin or ion exchange resin-drug solid particles, which employ a novel liquid suspending coating process based on a solid-in-oil-in water (S/O/W) emulsion/suspension method (S/O/W method).

The disclosed method is suitable for producing a coated micronized resinate that can be used directly or be incorporated in age-appropriate formulations to provide a completely grittiness-free mouthfeel while providing extended release features for the drug products or dosage forms, such as ER chewable, ER orally disintegrating tablet (ODT); ER film, or ER minitablet. The smaller size of the extended release coated micronized resinate can be better distributed in filler materials to produce a uniform drug product. It can be especially beneficial for a dosage form that requires uniformity in smaller size dosage units such as in minitablets. The coated micronized resinate as prepared by the disclosed methods can provide an added benefit for chewable tablets. Due to the micronized particle size, the extended release coated resinate reduces the risk of losing extended release feature due to coating damage caused by chewing.

The coated micronized resinate as prepared by the disclosed methods can be incorporated in liquid suspensions of age-appropriate formulations to reduce the uniformity risk during a manufacturing process such as filling and in the finished product. The age-appropriate extended release formulations are better suited for younger child patients such as infants and neonates in drug treatment, and can be added to infant food, such as milk or applesauce to aid drug administration while reducing dosing frequency.

In a coating process (also referred to a S/O/W emulsion/suspension method) according to various embodiments of this disclosure, an organic coating polymer solution is first prepared. Then, a drug-micronized ion exchange resin powder (micronized resinate) with known drug content is suspended in the coating polymer solution under agitation to produce a uniform suspension (first suspension). The first suspension is then added to a first aqueous solution containing a surfactant (first surfactant solution) under agitation to form a Solid-in-Oil-in-Water emulsion/suspension (second suspension). This process produces an Oil-in-Water emulsion in which each oil droplet contains the drug-micronized ion exchange resin solid particles. The second suspension is then forced through a micro-opening to produce a micronized S/O/W emulsion/suspension (third suspension). The third suspension is then added into another aqueous solution containing a surfactant (second surfactant solution) under stirring. The resulting suspension is quenched at room temperature or heated temperature to evaporate/remove the organic solvent. The quenching step will cause the coating polymer to lose solvent and harden on the surface of the drug-micronized ion exchange resin particles. The S/O/W emulsion/suspension method produces mostly discrete coated drug-micronized ion exchange resin particles without significant agglomeration. Once the solvent removal is completed, the coated drug-micronized ion exchange resin particles are collected by filtration or centrifugation. The wet collected drug-micronized ion exchange resin particles are then dried.

The methods as disclosed can be used to coat micronized ion-exchange resin or ion-exchange resin-drug complex particles. The coated micronized resinate possesses an extended release characteristic for the pharmaceutically active agent and a grittiness-free mouthfeel. The coated micronized resinate is useful for providing an extended release characteristic to many age-appropriate pharmaceutical formulations such as oral suspension, orally disintegrating tablet (ODT), chewable tablet, minitablet, and oral film without bringing a gritty mouthfeel to these formulations. These pharmaceutical formulations target special patient populations such as pediatric, geriatric patients, or patients who require enteral drug administration.

In one aspect, this disclosure provides a method of coating ion-exchange resin particles. In some embodiments, the method comprises: (a) dissolving a coating polymer in a solvent to form a first solution; (b) suspending ion-exchange resin particles uniformly in the first solution to obtain a solid-in-oil suspension; (c) adding the solid-in-oil suspension to a second solution that contains a surfactant and is immiscible with the first solution to obtain a solid-in-oil-water suspension; (d) mixing the solid-in-oil-water suspension using agitation to form a solid-in-oil-in water emulsion/suspension (S/O/W dispersion); (e) passing the S/O/W dispersion through a mechanism or device that exerts a pressure and/or shear force on the S/O/W dispersion and sending resulting dispersion into a third solution that contains a surfactant to obtain a micronized S/O/W dispersion; (f) removing the solvent from the micronized S/O/W dispersion to deposit and harden the coating polymer on the ion-exchange resin particles to obtain coated ion-exchange resin particles; and (g) collecting and drying the coated ion-exchange resin particles.

In some embodiments, the method comprises: (i) dissolving a coating polymer in a solvent to product a Coating Polymer Solution; (ii) suspending a micronized ion exchange resin-drug complex in the Coating Polymer Solution to produce an Organic Suspension; (iii) dissolving a surfactant in water to produce a First Surfactant Solution; (iv) adding the Organic Suspension into the First Surfactant Solution under agitation to produce a Solid-in-Oil-in Water (S/O/W) Suspension/Emulsion; (v) dissolving a surfactant in water to produce a Second Surfactant Solution; (vi) passing the Solid-in-Oil-in Water (S/O/W) Suspension/Emulsion Complex through an orifice with the proper size opening to reduce the droplet size; (vii) collecting the Solid-in-Oil-in Water (S/O/W) Suspension/Emulsion into the Second Surfactant Solution under stirring; (viii) continuing stirring the Solid-in-Oil-in Water (S/O/W) Suspension/Emulsion to evaporate the organic solvent to an acceptable level and allow the coating film to solidify to form coated resinate particles; and (ix) collecting the coated resinate particles and dry, wherein the coated resinate particles have a particle size of D90 less than 30-50 microns.

A coating polymer solution is prepared by dissolving a coating polymer in a suitable solvent to obtain an organic polymer solution. A suitable solvent should provide sufficient solubility for the coating polymer and have limited solubility in water. In some embodiments, solvents that have a water solubility of about 10 grams per 100 mL of water or less and above about 0.08 grams per 100 mL of water at 25° C. temperature can be used. Non-limiting examples of these solvents include dichloromethane, ethyl acetate, tetrahydrofuran, 1,2-dichloroethane, diethyl ether, and carbon tetrachloride. The concentration of the coating polymer solution used depends on the desired coating amount and the polymer solubility in the selected solvents. In some embodiments, the concentration of the coating polymer solution is in the range of from about 1% to about 30% (e.g., 1.0%, 1.2%, 1.4%, 1.6%, 1.8%, 2.0%, 2.2%, 2.4%, 2.6%, 2.8%, 3.0%, 3.2%, 3.4%, 3.6%, 3.8%, 4.0%, 4.2%, 4.4%, 4.6%, 4.8%, 5.0%, 5.2%, 5.4%, 5.6%, 5.8%, 6.0%, 6.2%, 6.4%, 6.6%, 6.8%, 7.0%, 7.2%, 7.4%, 7.6%, 7.8%, 8.0%, 8.2%, 8.4%, 8.6%, 8.8%, 9.0%, 9.2%, 9.4%, 9.6%, 9.8%, 10.0%, 10.2%, 10.4%, 10.6%, 10.8%, 11.0%, 11.2%, 11.4%, 11.6%, 11.8%, 12.0%, 12.2%, 12.4%, 12.6%, 12.8%, 13.0%, 13.2%, 13.4%, 13.6%, 13.8%, 14.0%, 14.2%, 14.4%, 14.6%, 14.8%, 15.0%, 15.2%, 15.4%, 15.6%, 15.8%, 16.0%, 16.2%, 16.4%, 16.6%, 16.8%, 17.0%, 17.2%, 17.4%, 17.6%, 17.8%, 18.0%, 18.2%, 18.4%, 18.6%, 18.8%, 19.0%, 19.2%, 19.4%, 19.6%, 19.8%, 20.0%, 20.2%, 20.4%, 20.6%, 20.8%, 21.0%, 21.2%, 21.4%, 21.6%, 21.8%, 22.0%, 22.2%, 22.4%, 22.6%, 22.8%, 23.0%, 23.2%, 23.4%, 23.6%, 23.8%, 24.0%, 24.2%, 24.4%, 24.6%, 24.8%, 25.0%, 25.2%, 25.4%, 25.6%, 25.8%, 26.0%, 26.2%, 26.4%, 26.6%, 26.8%, 27.0%, 27.2%, 27.4%, 27.6%, 27.8%, 28.0%, 28.2%, 28.4%, 28.6%, 28.8%, 29.0%, 29.2%, 29.4%, 29.6%, 29.8%, 30.0%, or any intermediate values therebetween) by weight. In some embodiments, the concentration of the coating polymer solution is in the range of from about 3% to about 25% by weight, e.g., from about 5% to about 20% by weight. In some embodiments, a single solvent can be employed for making the coating polymer solution. In some embodiments, a mixture of two or more solvents can be mixed to create a co-solvent for making the coating polymer solution.

In some embodiments, a plasticizer can be added to the coating polymer solution to improve the flexibility of the polymer. It is necessary when the coating polymer itself does not have sufficient flexibility. Polymer flexibility may be important when coating small particles like micronized drug-ion exchange complexes due to higher curvatures on the surface. The ratio of the plasticizer to coating polymer is about 1:20 (e.g., 1:1, 1:1.5, 1:2, 1:2.3, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5, 1:10, 1:10.5, 1:11, 1:11.5, 1:12, 1:12.5, 1:13, 1:13.5, 1:14, 1:14.5, 1:15, 1:15.5, 1:16, 1:16.5, 1:17, 1:17.5, 1:18, 1:18.5, 1:19, 1:19.5, 1:20 or any intermediate values therebetween). In some embodiments, the ratio of the plasticizer to coating polymer ranges from about 1:5 to 1:15.

The drug-micronized ion exchange resin particles are dispersed in the coating polymer solution under agitation to form a solid-in-oil suspension (S/O suspension). In one embodiment, the agitation can be brought about by a mechanical mixer or homogenizer. In another embodiment, the agitation can be brought about by sonication.

The ratio of drug-micronized ion exchange resin particles to coating polymer solution used depends on the target drug release profile and the amount of coating applied. The percent concentration of drug-micronized ion exchange resin particles in the resulting S/O suspension ranges from about 1% to 50% (e.g., 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or any intermediate values therebetween) by weight, e.g., from about 10% to 40% by weight, from about 15% to about 35% by weight.

The S/O suspension is further dispersed into an aqueous surfactant solution under agitation to form a solid-in-oil-in-water emulsion (S/O/W emulsion), wherein the drug-micronized ion exchange resin particles are suspended in the droplets of organic polymer solution and in the meantime the droplets are emulsified in the aqueous surfactant solution.

The amount of surfactants used in the solution should be sufficient to allow the formation of a relatively stable emulsion with suitable droplet size and free of significant agglomerations, but not too much to cause the majority of the polymer solution to form fine droplets by itself. The S/O/W emulsion should possess necessary physical stability to go through the subsequent process. The surfactant solution has a concentration range from 0.5% to 10% (e.g., 0.50%, 1%, 1.50%, 2%, 2.50%, 3%, 3.50%, 4%, 4.50%, 5%, 5.50%, 6%, 6.50%, 7%, 7.50%, 8%, 8.50%, 9%, 9.50%, 10%, or any intermediate values therebetween) by weight, e.g., from 0.8% to 5%, from 1% to 3%.

In some embodiments, the surfactants can be non-ionic surfactants that do not interact with the ion exchange resins or the drug-ion exchange reason particles. Non-limiting examples of non-ionic surfactants include polymeric surfactants like polyvinyl alcohol, poloxamers (Pluronics), polysorbates (Tween), high molecular weight polyvinylpyrrolidone, polyoxyethylene stearate (Solutol HS), Polyoxyethylene lauryl ether (Brij).

In some embodiments, the weight ratio of S/O suspension to the surfactant solution ranges from about 1:10 to about 1:100 (e.g., 1:10, 1:12, 1:14, 1:16, 1:18, 1:20, 1:22, 1:24, 1:26, 1:28, 1:30, 1:32, 1:34, 1:36, 1:38, 1:40, 1:42, 1:44, 1:46, 1:48, 1:50, 1:52, 1:54, 1:56, 1:58, 1:60, 1:62, 1:64, 1:66, 1:68, 1:70, 1:72, 1:74, 1:76, 1:78, 1:80, 1:82, 1:84, 1:86, 1:88, 1:90, 1:92, 1:94, 1:96, 1:98, 1:100, or any intermediate values therebetween), e.g., from about 1:15 to about 1:50, from about 1:20 to about 1:40.

In some embodiments, the agitation is brought about by a homogenizing mixer. The homogenizer works by applying high shear mechanical force to reduce a droplet size of a dispersion. In one embodiment, the homogenizing mixer is equipped with a high-shear rotor-stator assembly component. The rotor-stator assembly component has a rotor and a stator element. The rotor is a rotating element typically equipped with blades or pins, while the stator is a stationary component with corresponding slots or grooves. The rotor and stator are closely spaced, creating a narrow gap between them. When the rotor rotates, it induces high shear forces and turbulence in the fluid passing through the narrow gap between the rotor and stator, reducing the droplet size in the passing fluid.

The addition of the S/O suspension to the surfactant solution can be through a mechanism in which the suspension is sent through an opening with defined dimension. In some embodiments, the suspension is delivered to the immediate premixes of the rotor-stator assembly component of the homogenizing mixer to produce consistent droplet size in each processing batch.

In some embodiments, the droplet size produced in the S/O/W emulsion is in 20-1000 microns range (e.g., 20 microns, 40 microns, 60 microns, 80 microns, 100 microns, 120 microns, 140 microns, 160 microns, 180 microns, 200 microns, 220 microns, 240 microns, 260 microns, 280 microns, 300 microns, 320 microns, 340 microns, 360 microns, 380 microns, 400 microns, 420 microns, 440 microns, 460 microns, 480 microns, 500 microns, 520 microns, 540 microns, 560 microns, 580 microns, 600 microns, 620 microns, 640 microns, 660 microns, 680 microns, 700 microns, 720 microns, 740 microns, 760 microns, 780 microns, 800 microns, 820 microns, 840 microns, 860 microns, 880 microns, 900 microns, 920 microns, 940 microns, 960 microns, 980 microns, 1000 microns, or any intermediate values therebetween), e.g., from 50 to 500 microns, from 80 to 300 microns.

The resulting S/O/W emulsion is further processed by a size reduction step to reduce the droplet size below about 30 microns. This is achieved by sending the S/O/W emulsion through a precisely engineered opening under pressure. In one embodiment, the size reduction process is done by sending the S/O/W emulsion through a microfluidizer. Microfluidizer is a high-pressure fluid processing technology used for the precise and controlled size reduction purposes. The mean components of a microfluidizer include high-pressure pump, sample reservoir, pressure chamber, in-line microchamber with a defined opening, and sample collection reservoir. The S/O/W emulsion is added to the sample reservoir. The high-pressure pump will drive the S/O/W emulsion through the in-line microfluidizer chamber with defined opening, forcing the droplets to reduce the size determined primarily by the micro-opening in the chamber and the pressure applied. In one embodiment, the size of the micro-opening ranges from 1 microns to 500 microns (e.g., 1 micron, 5 microns, 10 microns, 15 microns, 20 microns, 25 microns, 30 microns, 35 microns, 40 microns, 60 microns, 80 microns, 100 microns, 120 microns, 140 microns, 160 microns, 180 microns, 200 microns, 220 microns, 240 microns, 260 microns, 280 microns, 300 microns, 320 microns, 340 microns, 360 microns, 380 microns, 400 microns, 420 microns, 440 microns, 460 microns, 480 microns, 500 microns, or any intermediate values therebetween), e.g., from 1 to 200 microns, from 5 to 50 microns.

The pressure used for the process should be sufficient to produce the desired droplet size but not too high to strip a coating solution off the micronized drug-resin particles. The desired droplet size is the size that will lead to the formation of coated drug-micronized ion exchange resin particles in the D90 less than 30-50 microns. In one embodiment, the processing pressure ranges from 500 psi to 20000 psi (e.g., 500 psi, 1000 psi, 1500 psi, 2000 psi, 2500 psi, 3000 psi, 3500 psi, 4000 psi, 4500 psi, 5000 psi, 5500 psi, 6000 psi, 6500 psi, 7000 psi, 7500 psi, 8000 psi, 8500 psi, 9000 psi, 9500 psi, 10000 psi, 10500 psi, 11000 psi, 11500 psi, 12000 psi, 12500 psi, 13000 psi, 13500 psi, 14000 psi, 14500 psi, 15000 psi, 15500 psi, 16000 psi, 16500 psi, 17000 psi, 17500 psi, 18000 psi, 18500 psi, 19000 psi, 19500 psi, 20000 psi, or any intermediate values therebetween), e.g., from 3000 psi to 15000 psi, from 5000 psi to 10000 psi.

In some embodiments, the S/O/W emulsion size reduction step can be achieved by sending the S/O/W emulsion through an in-line homogenizer with a design similar to IKA UTL 25 digital Inline ULTRA-TURRAX®, in which an in-line mixing chamber is attached to the homogenizer head. In some embodiments, the S/O/W emulsion size reduction can be achieved by a single pass through the homogenizer. In some embodiments, the S/O/W emulsion size reduction can be achieved by multiple passes through the homogenizer. The speed used for the in-line homogenizer varies in the range of 500 to 20,000 rpm depending on the size of the homogenizer and the volume of materials being processes.

After the size reduction step, the micronized S/O/W emulsion/suspension is transferred into a larger volume of a second surfactant solution for quenching. The quenching process removes the solvent and hardening the coating polymer. The quenching suspension is continuously stirred by a mixer for a duration that allows the removal of the solvent to an acceptable level. The typical acceptable level of residual solvents is defined in the relevant chapters or monographs by pharmacopeias such as United States Pharmacopeia (USP), European Pharmacopoeia (Ph. Eur.), or Chinese Pharmacopoeia (ChP).

The surfactant concentration of the second surfactant solution is lower than the first surfactant solution. In one embodiment, the surfactant solution has a concentration range from 0.1% to 5% (e.g., 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, or any intermediate values therebetween), e.g., from 0.2% to 3, from 0.3% to 1%.

In some embodiments, quenching can be performed at room temperature. The suspension being quenched is continuously stirred until the solvent is removed to an acceptable level. The quenching also sets the coating polymer to a stabilized state that gives the final product a stable drug release profile over its shelf life. In some embodiments, heating can be used to shorten the solvent removal time. Heating the quenching suspension to a higher temperature can speed up solvent removal and cure the coating polymer. The temperature employed depends on the stability of the product and the drug. The effect of the polymer hardening on the drug release behavior also affects the temperature selected. In one embodiment, the temperature of the quenching suspension can be from about 25° C. to 80° C. (e.g., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., or any intermediate values therebetween), e.g., from about 30 to 60° C., from about 35-45° C. In yet another embodiment, a vacuum can be applied to facilitate the solvent removal from the quenching suspension.

Once the quenching is completed, the coated micronized drug-ion exchange resin particles are collected for subsequent uses. In one embodiment, the coated micronized drug-ion exchange resin particles can be filtration collected. In another embodiment, the coated micronized drug-ion exchange resin particles can be centrifuged and collected. In another embodiment, the coated micronized drug-ion exchange resin particles can first be centrifuged to remove most of the water, and then collected by filtration.

The wet collected coated micronized drug-ion exchange resin particles can be used directly as a component to be further formulated into various suitable age-appropriate dosage forms described above.

The wet collected coated micronized resinate particles can also be dried first before being used as a component to be further formulated into various suitable age-appropriate dosage forms described above. The collected wet coated micronized drug-ion exchange resin particles can be dried by different drying methods known in the art, including oven drying, fluid bed drying, spray drying, and freeze-drying. Pharmaceutically acceptable adjuvants can be added to the collected coated micronized drug-ion exchange resin particles to serve as dispersing agents before drying. The adjuvants facilitate the dispersion of the coated micronized drug-ion exchange resin particles in a subsequent use. In one embodiment, the adjuvants can be various grades of water-soluble pharmaceutical excipients that do not interfere with the polymer coating or the drug-ion exchange resin complexation. Examples include water soluble sugars such as lactose, mannitol, sucrose, sorbitol, dextrose, maltose, trehalose, xylitol, maltitol, isomalt, or a mixture thereof. In another embodiment, the adjuvants can be micronized insoluble materials (D90<30 microns) that provide a physical barrier between the coated drug-micronized ion exchange resin particles. Examples are micronized fillers or disintegrants such as microcrystalline cellulose, talc, starch, silicone oxide, sodium starch glycolate, and crospovidone.

In one embodiment, the dried coated micronized drug-ion exchange resin particles can also be used as an age-appropriate dosage form by itself, such as oral powder, or powder for oral liquid dosage forms. In another embodiment, the dried coated micronized drug-ion exchange resin particles can be used as an intermediate component to be added to formulations of other age-appropriate dosage forms described above.

### Components of Coated Drug Ion-Exchange Resin Complexes

### Ion-Exchange Resins

Ion-exchange resins suitable for use in these preparations are water-insoluble and comprise a preferably pharmacologically inert organic and/or inorganic matrix containing functional groups that are ionic or capable of being ionized under the appropriate conditions of pH. The organic matrix may be synthetic (e.g., polymers or copolymers of acrylic acid, methacrylic acid, sulfonated styrene, sulfonated divinylbenzene), or partially synthetic (e.g., modified cellulose and dextrans). The inorganic matrix may include silica gel modified by the addition of ionic groups. Covalently bound ionic groups may be strongly acidic (e.g., sulfonic acid, phosphoric acid), weakly acidic (e.g., carboxylic acid), strongly basic (e.g., primary amine), weakly basic (e.g., quaternary ammonium), or a combination of acidic and basic groups. In general, the types of ion exchangers suitable for use in ion-exchange chromatography and for such applications as deionization of water are suitable for use in the present disclosure. Such ion-exchangers are described by H. F. Walton in “Principles of Ion Exchange” (pp: 312-343) and “Techniques and Applications of Ion-Exchange Chromatography” (pp: 344-361) in Chromatography. (E. Hoffmann), van Nostrand Reinhold Company, New York (1975). Ion exchange resins that can be used in the present invention have exchange capacities of about 6 milliequivalents (meq)/gram or below (e.g., about 5.5 meq/gram, about 5 meq/gram, about 4.5 meq/gram, about 4 meq/gram, about 3.5 meq/gram, about 3 meq/gram).

Suitable ion-exchange resins include anion exchange resins, such as have been described in the art and are commercially available. These resins are particularly well suited for use with acidic drugs including, e.g., nicotinic acid, mefenamic acid, indomethacin, diclofenac, repaglinide, ketoprofen, ibuprofen, valproic acid, lansoprazole, ambroxol, omeprazole, acetaminophen, topiramate, and carbamazepine, pentobarbital, warfarin, triamterene, and prednisolone, as well as prodrugs, salts, isomers, polymorphs, and solvates thereof, as well as other drugs identified herein and/or known in the art.

An example of an anion exchange resin is a cholestyramine resin, a strong base type 1 anion exchange resin powder with a polystyrene matrix and quaternary ammonium functional groups. The exchangeable anion is generally chloride which can be exchanged for, or replaced by, virtually any anionic species. A commercially available Cholestyramine resin is PUROLITE™ A430MR resin. As described by its manufacturer, this resin has an average particle size range of less than 150 μm, a pH in the range of 4-6, and an exchange capacity of 1.8-2.2 eq/dry gm. Another pharmaceutical-grade cholestyramine resin is available as DUOLITE™ AP143/1094, described by the manufacturer as having a particle size in the range of 95%, less than 100 microns and 40%, less than 50 microns. The commercial literature from the suppliers of these and other resin is incorporated herein by reference (PUROLITE A-430 MR; DOW Cholestryramine USP, Form No. 177-01877-204, Dow Chemical Company; DUOLITE AP143/1083, Rohm and Haas Company, IE-566EDS). Another example of basic resins useful in this invention includes anion exchange resin such as Duolite AP143/1093 (colestyramine resin). Duolite AP143/1093 has a sodium glycocholate exchange capacity of 1.8-2.2 meq/g and a particle size range of no less than 95% less than 100 microns and no less than 40% less than 50 microns.

Cation exchange resins (e.g., AMBERLITE IRP-69) are well suited for use with drugs and other molecules having a cationic functionality, including, e.g., acycloguanosine, tinidazole, deferiprone, cimetidine, oxycodone, remacemide, nicotine, morphine, guanfacine, hydrocodone, rivastigmine, dextromethorphan, propanolol, betaxolol, 4-aminopyridine, chlorpheniramine, paroxetine, duloxetine HCl, atomoxetine HCl, risperidone, atovaquone, oseltamivir, esmolol, naloxone, phenylpropanolamine, gemifloxacin, oxymorphone, hydromorphone, nalbuphine, and O-desmethylvenlafaxine, as well as prodrugs, salts, isomers, polymorphs, and solvates thereof, as well as other drugs identified herein and/or known in the art. Cationic exchange resins are readily selected for the use of these basic drugs or other drugs identified herein and/or are those which are known to those of skill in the art.

Representative acidic resins useful in this invention include pharmaceutical-grade strongly acidic cation exchange resin such as AMBERLITE IRP-69 (sodium polystyrene sulfonate), and weakly acidic cation exchange resin Amberlite IRP-64 (polymethacrylic acid), obtained from Rohm and Haas/Dow. Amberlite IRP-69 has an ion exchange capacity of about 110 to 135 mg/g potassium and a particle size range of 10.0-25.0% larger than 75 microns and no more than 1.0% larger than 150 microns. Amberlite IRP-64 has an ion exchange capacity of about 10.0 meq/g and a particle size range of <=1.0% larger than 150 microns, 15.0-30.0% larger than 75 microns, and <=70.0% smaller than 50 microns. Its exchange capacity is normally within the range of approximately 3 to 4 meq/g of dry resin.

The selected ion-exchange resins may be further treated by the manufacturer or the purchaser to maximize the safety for pharmaceutical use or for improved performance of the compositions. Impurities present in the resins may be removed or neutralized by the use of common chelating agents, antioxidants, preservatives such as disodium edetate, sodium bisulfite, and so on by incorporating them at any stage of preparation either before complexation or during complexation or thereafter. These impurities along with their chelating agent to which they have bound may be removed before further treatment of the ion exchange resin with a release retardant and diffusion barrier coating.

In some embodiments, the resin particle is an anionic ion-exchange resin particle or a cationic ion-exchange resin particle. The resin particle can be a cross-linked sulfonated polystyrene ion-exchange resin (e.g., Amberlite IRP-69), a cross-linked methacrylic acid and divinylbenzene copolymer ion-exchange resin (e.g., Amberlite IRP-64), a cross-linked copolymer of diethylenetriamine and 1-chloro-2, 3-epoxy propane ion-exchange resin (e.g., Colestipol hydrochloride), or a cross-linked copolymer of styrene and divinylbenzene with quaternary ammonium functionality ion-exchange resin (e.g., Duolite AP143/1093). In some embodiments, the resin particle is Amberlite IRP-69, Amberlite IRP-64, Colestipol hydrochloride, or Duolite AP143/1093.

In some embodiments, the resin particle has an ion-exchange capacity of less than 6 (e.g., less than 5, less than 4, less than 3, less than 2) milliequivalents per gram (meq/g) of dry resin. Additional examples of commercially available ion-exchange resins and their trade names and grades include:

Dowex—Dowex resins are produced by Dow Chemical Company and are used for a variety of applications, including water purification, pharmaceutical manufacturing, and food processing. Some examples of Dowex resins include Dowex 50, Dowex Marathon C, and Dowex Optipore L-493.

Amberlite—Amberlite resins are produced by Rohm and Haas Company and are used for water treatment, chemical processing, and other industrial applications. Some examples of Amberlite resins include Amberlite IR-120, Amberlite XAD, and Amberlite XE-305.

Purolite—Purolite resins are produced by Purolite Corporation and are used for water treatment, pharmaceutical manufacturing, and other applications. Some examples of Purolite resins include Purolite C100, Purolite C900, and Purolite A520E.

Lewatit—Lewatit resins are produced by Lanxess Corporation and are used for water treatment, chemical processing, and other industrial applications. Some examples of Lewatit resins include Lewatit S-100, Lewatit MonoPlus M600, and Lewatit TP207.

Ionac—Ionac resins are produced by Ionac Chemical Company and are used for water treatment and chemical processing applications. Some examples of Ionac resins include Ionac C-249, Ionac A-249, and Ionac NM-60.

Commercially available ion-exchange resins have a particle size ranging from 50 microns to 1200 microns depending on the type and grade. Typically, these ion-exchange resins have a 90^{th} percentile particle size range of 120 to 200 microns. These ion-exchange resins can be coated by the invention as is or be micronized to a particle size 90^{th} percentile below about 30 microns, measured by dynamic light scattering or laser diffraction techniques, to obtain a grittiness-free mouthfeel from a resinate or coated resinate.

Typically, the ion-exchange resin or ion-exchange resin-drug complex particles need to be micronized first before being coated by the methods described in this invention. The commercial ion-exchange resins are first micronized to a particle size of D90 below 30 microns. This is necessary to ensure the coated micronized resinate has a particle size of D90 less than 30-50 microns. Particles below this size range will have a complete grittiness-free mouthfeel. The micronization of ion-exchange resins is achieved by many processes known in the art. Milling is the most common way for size reduction. Milling may be conducted in its dry state (dry milling) or suspended in a liquid medium (wet milling).

In one embodiment, a dry milling process is used. Resin particles can be milled using a jet mill. High velocity compressed air streams are injected into a chamber where the resin powder is fed by a rate-controlled feeder. As the resin particles enter the air stream, they are accelerated and caused to collide with each other and the wall of the milling chamber with high velocities. Particle size reduction is brought about by a combination of impact and attrition. Impacts arise from collisions between the rapidly moving particles and particles onto the wall of the milling chamber. Attrition occurs at surfaces of particles as they move rapidly against each other or the wall of the milling chamber, resulting in shear forces that break them up. The jet air pressure and milling time are adjusted for different types of resins to achieve desired particle sizes. The milling continues until D90 of the micronized resin is below 30 microns. The micronized resins are collected for subsequent drug-resin complexation process.

In another embodiment, a wet milling process is used. In some embodiments, the wet milling is done using a microfluidizer. In some embodiments, the wet milling is done by using a media mill. Resin powder is first suspended in an aqueous medium to produce a suspension. The aqueous medium serves as a carrier and a media to dissipate heat. In one embodiment, the medium is purified water. In other embodiments, adjuvants are added to water to serve as suspending, stabilization, or lubrication agents. These adjuvants are inert, non-toxic pharmaceutical excipients. They are typically water-soluble polymers, polymeric surfactants. Examples are polyvinylpyrrolidone, water soluble cellulose derivatives, polyethylene glycol, polyvinyl alcohol, poloxamers, natural polymers such as gelatin, Arabic gum, and non-ionic surfactants. These adjuvants can be used individually or in combination. The solid content of resin in the medium ranges from 1% to 50% wt.

In some embodiments, microfluidizer or high pressure/piston-gap homogenization wet milling process is used. In such processes, the suspension is added to a microfluidizer feeding reservoir under stirring. The suspended resin particles are forced under high pressure (a few thousand to tens of thousands psi) through precisely engineered microchannels or orifices. The powerful cavitation forces arising from the formation and collapse of the gas bubbles, coupled with an intense shearing effect, cause the resin to fracture into smaller particle sizes.

The solid content of the resin suspension should be appropriate for the processes. High solid content will generate more shear and friction among particles and improve the processing efficiency. However, too high of a solid content may cause excessive heat or cause the microchannel or orifice to clog. On the other hand, too low of a solid content can greatly reduce the processing efficiency. In some embodiments, cooling is applied to prevent the temperature to rise to a level that causes resin instability.

The resin particles will cycle through the microchannels or orifices repeatedly until the D90 of the resin particle size is below 30 microns. In some embodiments, the micronized resin particles are filter-collected and dried for subsequent drug-resin complexation process. In some embodiments, the micronized resin suspension is directly used in subsequent drug-resin complexation process.

In yet another embodiment, a wet media milling process is used. In such process, the resin particles are suspended in an aqueous solution. The suspension is mixed with a milling media constructed out of a variety of materials such as glass (yttrium-stabilized), zirconium oxide, or ceramics. The mixture is pumped through a milling chamber where the movement of the milling media is brought about by a disc mounted on a central shaft rotating at high velocities of 20,000 rpm and above. The micronized resin particles that have D90 of the resin particle size below 30 microns is screen separated from the milling media.

Once micronized below the limit of D90 of the resin particle size 30 microns, the ion exchange resin can be complexed with a drug. Adsorption of a drug onto the ion exchange resin particles to form drug-resin complex can be performed by a technique, as described in U.S. Pat. Nos. 2,990,332 and 4,221,778, the relevant disclosures of which are incorporated herein by reference. In general, the pharmaceutically active agent is mixed with an aqueous suspension of the resin for a certain period of time. Adsorption of pharmaceutically active agents onto the resin may be detected by measuring a change in the pH of the reaction medium, or by measuring a change in concentration of pharmaceutically active agents.

In some embodiments, the active pharmaceutical ingredients (APIs) or drugs are dissolved in water or buffer to form a solution. In some embodiments, the drugs are dissolved in a water and solvent mixture, to form a solution. In some embodiments, the drugs are suspended in water or buffer to form a suspension. The solution or suspension pH may be adjusted to cause the drug salts to dissociate, dissolve, or partially dissolve. The micronized resin is added to the drug solution or suspension under stirring to initiate ion exchange or complexation. The drug concentration in the solution is measured periodically to determine the complexation end point. The pH of the solution may be adjusted to drive the complexation to completion.

Complexation of a drug with ion exchange resin is influenced by the inherent strength of ionic interaction of the ion exchange resin to the drug, the drug concentration in the exchange solution, and the counter ion concentration and the pH in the exchange solution. The concentration of the drug in the solution used depends on the balance of the drug solubility and the manufacturability of the process. Higher solubility drug allows higher concentration and smaller volume of the complexation liquid. A skilled person in the art can easily determine the appropriate drug concentration for the complexation according to the drug and ion exchange resin selected and the manufacturability of the processing equipment. In one embodiment, the drug concentration of about 0.1% to 10% w/v can be used. When a drug suspension is used in the complexation process, the complexation happens between the dissolved drug and the ion exchange resin used. In this process, the drug concentration in the exchange solution will continuously decrease by the complexation and replenish by the dissolving of the drug. The complexation time is usually longer than using the fully dissolved drug as a starting material. The drug solid content in the exchange solution can be experimentally determined and selected.

The ratio of API and micronized ion exchange resin used for the complexation depends on the selectivity of the resin for the drug, the ionic strength and the molecular weight of the drug, the dose required for the intended product, and the manufacturability of the production process. In one embodiment, the ratios can be in the range of about 1:1 to about 1:10 by weight. The drug content in the complexes typically ranges from about 1% to 50% by weight of the complex particles. In one embodiment, when the therapeutic drug dose is relatively low, the drug content in the complexes ranging from about 1% to 10% by weight of the complex particles can be used. In another embodiment, when the therapeutic drug dose is relatively high, the drug content in the complexes ranging from about 10% to 50% by weight of the complex particles can be used.

After complexation is completed, residual free drug is usually present in the exchange medium. The drug-ion exchange resin particles are washed to remove the free drug. In one embodiment, the drug-ion exchange resin particles are washed with purified water several times. The washed drug-ion exchange resin particles can be collected by filtration or centrifugation. The collected drug-ion exchange resin particles can be dried for further use. The drug content in the dried drug-ion exchange resin particles is typically determined by a chromatographic analytical method, in which the bounded drug is eluted from the micronized ion exchange resin particles by a buffer solution containing counter ions. The amount of drug eluted is determined by the chromatographic analytical method.

### Drugs

The coated micronized resinate is comprised of at least one therapeutically effective amount of a pharmaceutical active ingredient that is capable of ionically complexing with at least one micronized ion-exchange resin to form a resinate. The pharmaceutical active ingredients that are suitable for producing the resinate include all compounds that have groups that can exert an ionic interaction with the ion-exchange resins. These include pharmaceutical salts that can dissociate in a suitable media. Non-limiting examples of the pharmaceutical salts may include: acetate salts (e.g., Prednisolone Acetate), Trenbolone Acetate, Testosterone Acetate, Methylprednisolone Acetate; Benzoate salts (e.g., Amlodipine Benzoate); Calcium salts (Ca) (e.g., calcium carbonate and calcium gluconate); Carbonate salts (e.g., sodium carbonate and magnesium carbonate, which are used in antacid medications to neutralize stomach acid); Chloride salts (e.g., magnesium chloride and calcium chloride, which are used in intravenous fluids to replenish electrolytes); Citrate salts (e.g., potassium citrate and sodium citrate, which are used to treat kidney stones and metabolic acidosis, respectively); Fumarate salts (e.g., Quetiapine Fumarate, Tramadol Fumarate); Gluconate salts (e.g., chlorhexidine gluconate and calcium gluconate, which are used as antiseptics and to treat hypocalcemia, respectively); Gluconate salts (Glu) (e.g., iron gluconate and zinc gluconate); Hydrochloride salts (HCl) (e.g., Cetirizine Hydrochloride, Metformin Hydrochloride, Ranitidine Hydrochloride, Sertraline Hydrochloride, Lidocaine Hydrochloride, amitriptyline HCl, loratadine HCl, and tramadol HCl); Hydroxide salts (e.g., magnesium hydroxide and aluminum hydroxide, which are used in antacid medications to neutralize stomach acid); Lactate salts (e.g., sodium lactate and calcium lactate, which are used as electrolyte supplements and in dialysis solutions); Lysine salts (Lys) (e.g., ibuprofen lysine and cefuroxime axetil lysine); Magnesium salts (Mg) (e.g., magnesium hydroxide and magnesium citrate); Maleate salts (e.g., Enalapril Maleate, Chlorpheniramine Maleate, Methylergometrine Maleate, Amlodipine Maleate, Fexofenadine Maleate); Mesylate salts (e.g., imatinib mesylate and tosylate mesylate, Prazosin Mesylate, Risperidone Mesylate, Tamsulosin Mesylate); Nitrate salts (NO3) (e.g., silver nitrate and nitroglycerin); Oxalate salts (e.g., Escitalopram Oxalate, Phenylalanine Oxalate); Phosphate salts (PO4) (e.g., dexamethasone phosphate); Phosphonate salts; Potassium salts (K); Protamine salts (Prot); Sodium salts (Na); Succinate salts (e.g., sumatriptan succinate, metoprolol succinate); Sulfate salts (SO4) (e.g., albuterol sulfate, and morphine sulfate); Sulfonate salts (e.g., furosemide sodium and sulbactam sodium, which are used to treat edema and bacterial infections, respectively); Tartrate salts (e.g., metoprolol tartrate and tolterodine tartrate, Disopyramide Tartrate, Ergotamine Tartrate; Zinc salts (Zn).

### Coating Polymers

The micronized resinate is coated with a polymer to produce the desired modified release effects such as extended drug release effect or delayed release effect. The polymers used for coating micronized resinates to generate the extended release effects are water insoluble and water permeable synthetic or natural polymers. Examples of these polymers are:

Ethyl acrylate, methyl methacrylate, trimethylammonioethyl methacrylate copolymers with different monomer ratios (e.g., Eudragit RL, RS) or Ethyl acrylate, methyl methacrylate copolymer with different monomer ratios (e.g., Eudragit NM). They are commonly used for extended release coatings. These polymers are water-insoluble and can be formulated to provide a range of release profiles.

Cellulose derivatives—Cellulose derivatives such as ethylcellulose (EC), and methylcellulose (MC) are commonly used for extended release coatings. These polymers are water-insoluble and can provide a barrier to drug release, allowing for controlled release over an extended period of time.

Cellulose acetate—Cellulose acetate is another cellulose derivative that can be used for extended release coatings. Like ethylcellulose, it is water-insoluble but can be made water-permeable by adjusting its molecular weight or degree of substitution.

Polyvinyl acetate—Polyvinyl acetate is a water-insoluble polymer that can be used for extended release coatings. It is often formulated with other excipients to control its permeability.

Polyethylene-co-vinyl acetate (PEVA)—PEVA is a copolymer of polyethylene and vinyl acetate that can be used for extended release coatings. It is water-insoluble but can be made water-permeable by adjusting the ratio of polyethylene to vinyl acetate.

In one embodiment, Methacrylic acid, ethyl acrylate or methyl methacrylate copolymers are used for coating. In another embodiment, Methacrylic copolymers with trimethylammonioethyl methacrylate as a functional group known as Eudragit® RL or RS are used for giving a formulation an extended release effect.

### Plasticizer

In some embodiments, plasticizers can be added to the coating polymer solution to improve the flexibility. Examples of plasticizers that can be used include Triacetin (glycerol triacetate), Diethyl phthalate (DEP), Dibutyl phthalate (DBP), Acetyl tributyl citrate (ATBC), Triethyl citrate (TEC), Castor oil, or tributyl O-acetylcitrate.

### Other Adjuvants for Use in Coating

In some embodiments, other adjuvants can be added to the plasticized coating polymer to adjust the drug release behavior. When a higher drug release rate that cannot be obtained by the extended release coating polymer itself is desired, water-soluble materials that improve the permeability of the polymer coating can be added. These materials should be non-ionic materials such as sugars or water-soluble polymers, which dissolve in the aqueous phase of the coating process or dissolution medium, creating pores but do not ionically interfere with the complexation of the drug and ion exchange resin. These materials are sometimes referred to as “pore former” in the art. The addition of a pore former in the drug coating helps create channels or pores within the coating layer, which can influence the dissolution or release of the drug. By adjusting the type and amount of pore former used, pharmaceutical manufacturers can modify the release profile of the drug, such as achieving sustained release. In one embodiment, various sugars like lactose, sucrose, glucose, and mannitol can be used as water-soluble pore formers. In another embodiment, water-soluble polymers such as hydroxypropyl cellulose (HPC), hydroxyethyl cellulose (HEC), and polyvinylpyrrolidone (PVP) can be used.

In some embodiments, a pharmaceutically acceptable adjuvant can be added to the wet collected drug-micronized ion exchange resin particles before drying. The adjuvant is dried with the drug-micronized ion exchange resin particles and prevents the particles from binding to each other during drying. The adjuvants facilitate the dispersion of the drug-micronized ion exchange resin particles in the subsequent processing steps. In one embodiment, the adjuvants can be various grades of polyvinylpyrrolidone (PVP) or polyethylene glycol (PEG) or a mixture thereof. In another embodiment, the adjuvants can be micronized insoluble materials (D90<30 microns) that provide a physical barrier between the drug-micronized ion exchange resin particles. Examples are micronized talc, starch, sodium starch glycolate, and crospovidone.

### Other Components

In some embodiments, a formulation or dosage form containing coated drug ion-exchange resin complexes may include additional components, such as a sweetener, a pH agent, an antioxidant, a coloring agent, a flavoring etc.

A sweetener is a substance that provides a sweet taste in the mouth of a subject. In some embodiments, a sweetener can be sugar. Sugar is the generic name for sweet-tasting, soluble carbohydrates, many of which are used in food. Simple sugars, also called monosaccharides, may include glucose, fructose, and galactose. Compound sugars, also called disaccharides or double sugars, are molecules made of two monosaccharides joined by a glycosidic bond. Common examples are sucrose (glucose+fructose), lactose (glucose+galactose), and maltose (two molecules of glucose). Table sugar, granulated sugar, and regular sugar refer to sucrose, a disaccharide composed of glucose and fructose. Examples of natural sweeteners may include sucrose, glucose, fructose, D-ribose, galactose, maltose, maltitol, isomalt, mannitol, sorbitol, Xylitol, glycerol, Thaumatin, Glycyrrhizin, stevioside, and erythritol.

A sweetener can also be sugar alcohol. Sugar alcohols (also called polyhydric alcohols, polyalcohols, alditols or glycitols) are organic compounds, typically derived from sugars, containing one hydroxyl group attached to each carbon atom. They are white, water-soluble solids that can occur naturally or be produced industrially by hydrogenation of sugars. Since they contain multiple hydroxyl groups, they are classified as polyols. Examples of sugar alcohols may include xylitol, sorbitol, erythritol, hydrogenated starch hydrolysates (HSH), isomalt, lactitol, maltitol, and mannitol.

A sweetener can also be a sugar substitute or artificial sweetener. A sugar substitute is a substance that provides a sweet taste like that of sugar with higher intensity and containing significantly less food energy than sugar-based sweeteners. Artificial sweeteners may be derived through manufacturing of plant extracts or processed by chemical synthesis. Examples of artificial sweeteners may include neotame, sucralose, allulose, acesulfame potassium, advantame, alitame, aspartame, aspartame-acesulfame salt, cyclamate, neohesperidin DC, mogrosides, saccharin, steviol glycosides (stevia), and monk fruit.

In some embodiments, the formulation or dosage form may also include a pH-adjusting agent. The pH adjusting agents may be selected from acidic or basic compounds such as acetic acid, adipic acid, ammonium aluminum sulphate, ammonium, bicarbonate, ammonium carbonate, ammonium citrate, dibasic, ammonium citrate, monobasic, ammonium hydroxide, ammonium phosphate, dibasic, ammonium phosphate, monobasic, calcium acetate, calcium acid pyrophosphate, calcium carbonate, calcium chloride, calcium citrate, calcium fumarate, calcium gluconate, calcium hydroxide, calcium lactate, calcium oxide, calcium phosphate, dibasic, calcium phosphate, monobasic, calcium phosphate, tribasic, calcium sulphate, citric acid, fumaric acid, gluconic acid, hydrochloric acid, lactic acid, magnesium carbonate, magnesium citrate, magnesium fumarate, magnesium hydroxide, magnesium oxide, magnesium phosphate, magnesium sulphate, malic acid, phosphoric acid, potassium acid tartrate, potassium aluminum sulphate, potassium bicarbonate, potassium carbonate, potassium chloride, potassium citrate, potassium fumarate, potassium hydroxide, potassium lactate, potassium phosphate, dibasic, potassium phosphate, tribasic, potassium sulphate, sodium acetate, sodium bicarbonate, sodium bisulphate, sodium carbonate, sodium citrate, sodium hydroxide, sodium phosphate, dibasic, sodium phosphate, monobasic, sulphuric acid, tartaric acid, or a combination thereof.

In some embodiments, the composition can further contain an antioxidant for enhancing drug stability. The antioxidants may be selected from butylated hydroxyanisole, butylated hydroxytoluene, tert-butylhydroquinone, propyl gallate, erythorbic acid, ascorbyl palmitate, tocopherols, ethoxyquin, phosphates, ethylene diamine tetra acetic acid, tartaric acid, citric acid, lecithin, ascorbic acid, sulfites (as sulfur dioxide), ascorbyl stearate, or a combination thereof.

In some embodiments, the composition can contain a coloring agent selected from organic dyes or their lakes, natural colors, inorganic colors, or mineral colors. Dyes may be synthetic chemical compounds that exhibit certain colors. Examples of dyes may include tartrazine, erythrosine, sunset yellow, and patent blue v. lakes which are aluminum salts of FD&C water-soluble dyes extended on a substratum of alumina. FD&C lakes are largely water-insoluble forms of the common synthetic water-soluble dyes and are available in six basic colors: one yellow, one orange, two reds (a pink red and an orange red), two blues (a green-blue and a royal blue). They can be blended to create more lake colors as needed, including brown, green, orange, red, yellow, and purple. Examples may include aluminum lakes, brilliant blue lake, sunset yellow lake, amaranth lake, allura red lake, indigo, carmine lake, and quinoline yellow lake. Examples of natural colors or vegetable and animal colors are caramel, cochineal (a dried insect), carmine (the aluminum lake of the coloring matter of cochineal), riboflavin and anthocyanins, paprika oleoresin, beet root red, annatto, and curcumin. Examples of inorganic or mineral colors may include red and yellow ferric oxides and titanium dioxide.

In some embodiments, the composition can contain a flavoring agent. As used herein, the term “flavoring agents” may include those flavor ingredients known in the art, such as natural and artificial flavors. These flavoring agents may be chosen from synthetic flavor oils and flavoring ingredient aromatics and/or oils, oleoresins and extracts derived from plants, leaves, flowers, fruits, and so forth, and combinations thereof. Non-limiting representative flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil. Also, useful flavoring agents can be artificial, natural, and synthetic fruit flavors, such as vanilla and citrus oils, including lemon, orange, lime, grapefruit, yazu, sudachi, and fruit essences, including apple, pear, peach, grape, blueberry, strawberry, raspberry, cherry, plum, pineapple, watermelon, apricot, banana, melon, apricot, ume, cherry, raspberry, blackberry, tropical fruit, mango, mangosteen, pomegranate, papaya and so forth. Other potential flavors include a milk flavor, a butter flavor, a cheese flavor, a cream flavor, and a yogurt flavor; a vanilla flavor; tea or coffee flavors, such as a green tea flavor, a oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a camomile flavor, a mustard flavor, a cardamom flavor, a caraway flavor, a cumin flavor, a clove flavor, a pepper flavor, a coriander flavor, a sassafras flavor, a savory flavor, a Zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, and a wasabi (Japanese horseradish) flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor. These flavoring agents may be used in liquid or solid form and may be used individually or in admixture. Commonly used flavors include mints such as peppermint, menthol, spearmint, artificial vanilla, cinnamon derivatives, and various fruit flavors, whether employed individually or in admixture. Flavors may also provide breath-freshening properties, particularly the mint flavors when used in combination with cooling agents.

Other useful flavoring agents include aldehydes and esters such as cinnamyl acetate, cinnamaldehyde, citral diethylacetal, dihydrocarvyl acetate, eugenyl formate, p-methylamisol, and so forth may be used. Generally, any flavoring ingredient or food additive such as those described in Chemicals Used in Food Processing, publication 1274, pages 63-258, by the National Academy of Sciences, may be used. This publication is incorporated herein by reference.

Further examples of aldehyde flavoring agents include but are not limited to acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2 (berry fruits), tolyl aldehyde (cherry, almond), veratraldehyde (vanilla), 2,6-dimethyl-5-heptenal, i.e., melonal (melon), 2,6-dimethyloctanal (green fruit), and 2-dodecenal (citrus, mandarin), cherry, grape, strawberry shortcake, and mixtures thereof. These listings of flavoring agents are merely exemplary and are not meant to limit either the term “flavoring ingredient” or the scope of the invention generally.

In some embodiments, the flavoring ingredient may be employed in either liquid form and/or dried form. When employed in the latter form, suitable drying means such as spray drying the oil may be used. Alternatively, the flavoring ingredient may be absorbed onto water soluble materials, such as cellulose, starch, sugar, maltodextrin, gum arabic and so forth or may be encapsulated. The actual techniques for preparing such dried forms are well-known.

In some embodiments, the flavoring agents may be used in many distinct physical forms well-known in the art to provide an initial burst of flavor and/or a prolonged sensation of flavor. Without being limited thereto, such physical forms include free forms, such as spray dried, powdered, beaded forms, encapsulated forms, and mixtures thereof. In some embodiments, the flavoring agent comprises a strawberry flavoring agent. In some embodiments, the composition comprises from 0.27% w/w to 0.33% w/w (e.g., 0.270%, 0.275%, 0.280%, 0.285%, 0.290%, 0.295%, 0.300%, 0.305%, 0.310%, 0.315%, 0.320%, 0.325%, 0.330% (w/w)) of the flavoring agent, e.g., a strawberry flavor.

### Use of Coated Micronized Resinate

In some embodiments, the coated micronized resinate can be used to provide ER effect in various age-appropriate dosage forms with excellent mouthfeel, as further described below.

### ER Suspension

The coated micronized resin can be suspended in a suitable aqueous medium to form a grittiness-free modified release (extended release or delayed release) oral suspension. Oral suspension is one of the most commonly used age-appropriate dosage forms due to its advantage of flexible dosing. Ensuring content uniformity in oral suspensions is particularly important for medications with a narrow therapeutic index, meaning that there is a small difference between a safe and effective dose and a toxic dose. For these medications, even small variations in the amount of active ingredients can have significant clinical consequences. The small particle size of the coated micronized resin helps to reduce the risk of sedimentation in suspension and consequently reduces the risk of non-uniform and incorrect doses. Less risk of sedimentation also allows the use of a less viscous suspending medium in the oral suspension. Elderly patients with dysphagia may prefer less viscous liquid medications because they are easier to swallow. Less viscous liquid medications also make it easier to divide doses and reduce the risk of dose inaccuracy.

### ER ODT or Oral Film

The coated micronized resin can be incorporated in the formulation of orally disintegrating tablets to form a grittiness-free modified release (extended release or delayed release) orally disintegrating tablets or thin films. The small particle size of the coated micronized resin helps reduce the grittiness of the insoluble and modified release component in the tablet and film while promoting the uniform distribution of the active ingredients in these formulations. Due to the small particle size, the coated micronized resin can also reduce the risk of damage to the modified release coating caused by chewing action during ODT administration.

### ER Chewable Dosage Forms

The coated micronized resin can be incorporated in the formulation of chewable dosage forms, such as chewable tablets or capsules or the like, to form a grittiness-free modified release (extended release or delayed release) chewable dosage forms. The small particle size of the coated micronized resin helps reduce the grittiness of the insoluble and modified release components in these dosage forms and promotes the uniform distribution of the active ingredients in these dosage forms. Due to the small particle size, the coated micronized resin can also reduce the risk of damage to the modified release coating caused by chewing action during dosing.

### ER Minitab

Mini-tablets, including orally disintegrating mini-tablets are small, typically less than 3 mm in diameter and are designed to be easier for patients to swallow than traditional tablets. They can be administered directly to the child or mixed with food or liquid to make them easier to take. Mini tablets also offer dosing flexibility and can be useful in situations where precise dosing is important, such as in the case of chemotherapy or narrow therapeutic drugs. Formulating extended release minitablet is less practical than the traditional tablets. Matrix and osmotic pump extended release formulation technologies that work well for traditional tablets may not work for minitablets. Apply extended release coating on minitablets can produce extended drug release profiles. But coating of minitablets makes minitablets less effective as a pediatric dosage form. The coated minitablets cannot be formulated into orally disintegrating dosage forms because coated tablets will not disintegrate. The coated minitablets also give grainy mouthfeel when mixed with food for administration. When administered to younger children, accidental chewing may damage the coating and cause dose dumping.

Incorporating coated micronized resin can be an effective way of solving these problems. Coated micronized resin particles can be mixed in the blend of orally disintegrating minitablet formulations to provide extended drug release profiles and a grittiness-free mouthfeel due to their small particle size. In addition, the small particle of the coated micronized resin reduces the risk of drug content uniformity issues in minitablets. Content uniformity is crucial for minitablets when dosing flexibility is necessary and where precise dosing is important.

Other dosage form specific ingredients can be added to the above dosage forms besides the coated micronized resin. For oral suspension or powder for oral suspension, suspending agent, pH agent, flavoring agent, sweetener, or preservatives, etc., can be added to give the suspension necessary physical and chemical attributes related to physical stability (e.g., sedimentation), chemical stability (e.g., assay, impurities, dissolution, microbiology), and palatability (e.g., taste & flavor).

For solid dosage forms including ODT tablets, minitablets or mini ODT tablets,
fillers, disintegrants, lubricants can be added to provide acceptable physical form and fast disintegration characteristics. Sweeteners, flavoring agents, and pH agents can also be added for palatability and stability purposes. For chewable tablets, fillers/diluents are added to increase the bulk volume of the tablet. Gelling agents can be added to provide a desired degree of soft chewy texture. Sweeteners, flavoring agents, and pH agents can also be added for palatability and stability purposes. For chewable

For oral films, fillers and plasticizers can be added to provide acceptable shape and flexibility. Sweeteners, flavoring agents, and pH agents can also be added for palatability and stability purposes.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

The term “formulation,” in general, refers to a preparation that includes at least one pharmaceutically active compound optionally in combination with one or more excipients or other pharmaceutical additives for administration to a subject. In general, particular excipients and/or other pharmaceutical additives are typically selected with the aim of enabling a desired stability, release, distribution, and activity of active compound(s) for applications.

In general, pharmaceutically acceptable salts include, but are not limited to, chloride, bromide, iodide, nitrate, sulfate, bisulfate, phosphate, acid phosphate, isonicotinate, acetate, lactate, salicylate, citrate, tartrate, pantothenate, bitartrate, carbonate, ascorbate, succinate, maleate, gentisinate, fumarate, gluconate, glucuronate, saccharate, formate, carboxylate, benzoate, glutamate, sulfonate, methanesulfonate, ethanesulfonate, benzensulfonate, p-toluenesulfonate, selenate, and pamoate (i.e., 1,1′-methylene-bis-(2-hydroxy-3-naphthoate)) salts of compounds.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

The terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder, or pathological condition.

As used herein, the term “pharmaceutical grade” means that certain specified biologically active and/or inactive components in the drug must be within certain specified absolute and/or relative concentration, purity and/or toxicity limits and/or that the components must exhibit certain activity levels, as measured by a given bioactivity assay. Further, a “pharmaceutical grade compound” includes any active or inactive drug, biologic or reagent, for which a chemical purity standard has been established by a recognized national or regional pharmacopeia (e.g., the U.S. Pharmacopeia (USP), British Pharmacopeia (BP), National Formulary (NF), European Pharmacopoeia (EP), Japanese Pharmacopeia (JP), Chinese Pharmacopoeia (ChP) etc.). Pharmaceutical grade further incorporates suitability for administration by means including topical, ocular, parenteral, nasal, pulmonary tract, mucosal, vaginal, rectal, intravenous, and the like.

Doses are often expressed in relation to body weight. Thus, a dose which is expressed as [g, mg, or other unit]/kg (or g, mg etc.) usually refers to [g, mg, or other unit]“per kg (or g, mg etc.) bodyweight,” even if the term “bodyweight” is not explicitly mentioned.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method may include a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

Examples are provided below to demonstrate the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1

### Preparation of Micronized Ion-Exchange Resin Particles

Ion-exchange resins Amberlite IRP69, Amberlite IRP88, Amberlite IRP64, and Duolite AP143 were used in this study. These ion-exchange resins were micronized by methods similar to the illustration below.

About 5 to 50 g ion-exchange resins were suspended in 100-3000 mL purified water to form a suspension. The suspension was added to the hopper of a Microfluidizer (Microfluidics M-110P) equipped with a 200 μm and 87 μm nozzle chambers connected in sequence. The Microfluidizer pressure was set to 10,000 psi and was used to force the ion-exchange resin suspension through the chambers. The resin suspension was cycled through the microfluidizer continuously until the particle size of all particles was reduced below 30 μm, which was confirmed by a microscope. The micronized resin particles were collected by centrifuge and/or filtration and dried. Alternatively, the micronized resin particles suspended in the micronized medium can be used directly to complex with a drug.

The particle size distribution of the ion-exchange resins before and after the micronization were measured using a Malvern Particle Sizer (Model 3000) to illustrate the micronization outcome. A summary of the particle size measures before and after micronization is provided in Table 1.

TABLE 1
Particle size of ion-exchange resin before and after micronization (in μm)

| | IER Type | | | | | | | |
|---|---|---|---|---|---|---|---|---|
| | Amberlite IRP64 | | Amberlite IRP88 | | Amberlite IRP69 | | Duolite AP143 | |
| | (n = 5) | | (n = 5) | | (n = 5) | | (n = 5) | |
| Stage | Before | After | Before | After | Before | After | Before | After |
| Dv 10 | 22.0 | 6.0 | 23.6 | 3.5 | 30.8 | 2.6 | 8.9 | 3.5 |
| Dv 50 | 79.2 | 12.7 | 83.1 | 7.1 | 70.3 | 5.4 | 67.8 | 6.4 |
| Dv 90 | 141.6 | 27.9 | 163.0 | 12.2 | 122.2 | 9.6 | 200.2 | 10.2 |

As demonstrated in Table 1, the particle size (i.e., D90) of all micronized ion-exchange resins is below 30 microns after micronization.

Various micronized resin-drug complexes (resinates) were prepared in the examples provided below.

### Example 2

### Preparation of Micronized Resin—Guanfacine Complex Particles (Guanfacine Resinate)

30 g Amberlite IRP69 ion-exchange resin particles were suspended in 3000 mL purified water and micronized according to the method described in Example 1. Once the desired particle size were obtained, 14.92 g of guanfacine HCl was added under mechanical stirring. Continued mixing for 3 hours for the complexation to happen. The drug-micronized ion-exchange complex particles (i.e., guanfacine resinate) obtained were collected by centrifuge/filtration and dried. The dried particles were analyzed for drug content.

The particle size of the guanfacine resinate was measured using the Malvern 3000, and the results are shown in Table 2. The drug content in the resinate was analyzed by a HPLC method. The result is shown in Table 3.

TABLE 2
Particle size of Guanfacine Resinate (lot 8076-66)

| | Guanfacine Resinate |
|---|---|
| Sample Name | (lot 8076-66) |
| (Lot #) | (N = 5) |
| Dv 10 | 3.9 |
| Dv 50 | 11.7 |
| Dv 90 | 23.3 |

TABLE 3
Guanfacine concentration in Guanfacine Resinate

| Sample Name | Guanfacine Resinate |
|---|---|
| (Lot #) | (lot 8076-66) |
| Drug Concentration | 33.23 |
| in the Resinate (%) | |
| Measurement 1 | |
| Drug Concentration | 33.14 |
| in the Resinate (%) | |
| Measurement 2 | |
| Average | 33.18 |

### Example 3

### Preparation of Micronized Resin—Clonidine Complex Particles

1.2878 g of clonidine HCl was added to 1000 mL purified water under mechanical stirring, and mixing was continued until a clear solution was obtained. 10 g micronized Amberlite IRP64 ion-exchange resin particles prepared according to the method described in Example 1 were added to the clonidine solution under mechanical stirring for 3 hours. During complexation, 0.1 N NaOH solution was used to maintain pH to 6.0. The ion-exchange drug complex particles suspended in this solution were collected by centrifuge/filtration and freeze-dried. The dried particles were analyzed for drug content.

The particle size of the complex was measured by the Malvern 3000, and the results are shown in Table 4. The drug content in the resinate was analyzed by an HPLC method, and the results are shown in Table 5.

TABLE 4
Particle size of Clonidine Resinate (lot 9050-49)

| | Clonidine Resinate |
|---|---|
| Sample Name | (lot 9059-49) |
| (Lot #) | (N = 5) |
| Dv 10 | 6.1 |
| Dv 50 | 12.4 |
| Dv 90 | 23.9 |

TABLE 5
Clonidine Content in Clonidine Resinate

| Sample Name | Clonidine Resinate |
|---|---|
| (Lot #) | (lot 9059-49) |
| Drug Content in the | 8.93 |
| Resinate (%) | |

### Example 4

### Preparation of Micronized Viloxazine Resinate

10 g Amberlite IRP69 ion-exchange resin particles were added to 1000 mL purified water under mechanical stirring. The suspension was added to the hopper of a Microfluidizer (Microfluidics M-110P) equipped with a 200 μm and 87 μm nozzle chambers connected in sequence. The Microfluidizer pressure was set to 10,000 psi. The Microfluidizer was used to force the ion-exchange resin suspension through the chambers. The resin suspension was cycled through the microfluidizer continuously for 3.5 hours. The average particle size of the resin suspension was reduced below 25 μm and confirmed by a microscope. The actual average particle size was 22.73 μm. The Microfluidizer line was rinsed with 100 g purified water, and all micronized resin suspension was collected in a container.

11.54 g of Viloxazine HCl was added to the micronized resin suspension under mechanical stirring. Stirring was continued for 3 hours. The micronized ion-exchange drug complex particles were collected by centrifuge/filtration and dried. The dried particles were analyzed for drug content and the result is shown in Table 6.

TABLE 6
Viloxazine content in Viloxazine Resinate

| Sample Name | Viloxazine Resinate |
|---|---|
| (Lot #) | (lot 9059-87) |
| Drug Content in the Resinate | 48.30 |
| (%) Measurement 1 | |
| Drug Content in the Resinate | 48.59 |
| (%) Measurement 2 | |
| Average (%) | 48.45 |

### Example 5

### Preparation of Coated Micronized Guanfacine Resinate (8076-70)

1. Preparation of Coating Solution (Eudragit RL100, Solution 1).
   1013 mg Eudragit RL100 was dissolved it in 6000 mg Methylene Chloride (CH₂Cl₂) under mechanical mixing until a clear polymer solution is obtained.
2. Preparation of Resin-Polymer suspension:
   2002 mg Resin-Guanfacine resinate according to procedures described in Example 2 was dispersed uniformly in the above Eudragit RL100 Solution 1 (labeled as Suspension 1).
3. Prepare a 250 mL 1.8% Polyvinyl Alcohol (PVA) Aqueous Solution (PVA Solution 1).
4. Prepare a 2500 mL 0.5% PVA Aqueous Solution (PVA Solution 2):
5. Add 5 mL Suspension 1 to PVA Solution 1 while mixing using a homogenizer (PT-MR 3000) for 5 min at 10000 RPM (Dispersion 1).
6. Pass the Dispersion 1 through a Microfluidizer (Microfluidics M-110P) equipped with two nozzle chambers, replacement (front) and 87 μm (back), in serial order with a pressure of 6500 psi.
7. The resulting dispersion is discharged into PVA Solution 2 under mixing. Continue mixing for 2 hours at room temperature. Then raise the temperature to 35° C. and continue mixing for another 3 hours.
8. Collect the coated micronized Guanfacine resinate by centrifuging. Wash the collected coated Guanfacine resinate twice with purified water. Collect the washed coated micronized Guanfacine resinate by centrifuging again.
9. Dry the coated clonidine resinate using a freeze dryer.

The dried coated micronized Guanfacine resinate was tested for particle size distribution, drug content, and dissolution, etc. The results are provided in Tables 7 & 8.

TABLE 7
Particle size of Coated Guanfacine Resinate (lot 8076-70)

| | Guanfacine Resinate |
|---|---|
| Sample Name | (lot 8076-70) |
| (Lot #) | (N = 5) |
| Dv 10 | 7.2 |
| Dv 50 | 16.9 |
| Dv 90 | 39.9 |

TABLE 8
Drug Content of Guanfacine in Guanfacine resinate

| Sample Name | Coated Guanfacine Resinate |
|---|---|
| (Lot #) | (8076-70) |
| Drug content (%) | 30.15 |

The following dissolution method was used:

Dissolution medium: phase 1 (0.5, 1, & 2 hours): 500 ml 0.085N HCl solution containing 0.2% NaCl and Phase 2: after 2-hour time point, 400 ml pH 6.8 KH₂PO4 is added to phase 1 medium, 75 rpm, at 37 C, USP apparatus II.

The dissolution profile is shown in Table 9.

TABLE 9
Dissolution profile of coated micronized Guanfacine resinate 4 mg.

| Time (hour) | 8076-70 |
|---|---|
| 0.5 | 12 |
| 1 | 16 |
| 2 | 21 |
| 4 | 35 |
| 6 | 45 |
| 12 | 62 |
| 16 | 69 |
| 20 | 73 |
| 24 | 77 |

### Example 6

### Coating of Clonidine Resinate

### Micronized

In this example, non-micronized resinate was coated using the disclosed method. Dissolution profiles of coated resinate and un-coated resinate were compared to demonstrate the extended release effect produced by coating method by the disclosed method.

### Preparation of Coated Clonidine Resinate (9059-50).

1. Preparation of Coating Solution.
   1600 mg Eudragit RS100 was dissolved in 4571 mg Methylene Chloride (CH₂Cl₂) under mechanical mixing until a clear polymer solution was obtained (labeled as Eudragit RS100 Solution 1).
2. Preparation of Resin-Polymer suspension:
   2000 mg Amberlite IRP64 Resin-Clonidine resinate prepared according to procedures described in Example 3 was disperse uniformly in the above Eudragit RS100 Solution (labeled as Suspension 1).
3. Prepare a 250 mL 1.5% Polyvinyl Alcohol (PVA) Aqueous Solution (PVA Solution 1);
4. Preparation a 2500 mL 0.5% PVA Aqueous Solution (PVA Solution 2);
5. Add 5 mL Suspension 1 to PVA Solution 1 while mixing using a homogenizer (PT-MR 3000) for 5 min at 10000 RPM (Dispersion 1).
6. Pass the Dispersion 1 through the Microfluidizer (Microfluidics M-110P) equipped with two microchannel chambers, replacement (front) and 200 μm (back), in serial order with a pressure of 4000 psi.
7. The resulting dispersion is discharged into the PVA Solution 2 under mixing. Continue mixing for 2 hours at room temperature, then raise the temperature of the PVA Solution 2 to 35° C. and continue mixing for additional 2 hours.
8. Collect the coated Clonidine resinate by centrifuging. Wash the collected coated Clonidine resinate twice with purified water. Collect the washed coated Clonidine resinate by centrifuging again.
9. Dry the coated clonidine resinate using a freeze dryer.

The dried coated Clonidine resinate was tested for particle size distribution, drug content, dissolution, etc. The results are provided in Table 10 & 11 respectively.

TABLE 10
Particle size of Coated Clonidine Resinate (lot 9059-50)

| | Coated Clonidine Resinate |
|---|---|
| Sample Name | (lot 9059-50) |
| (Lot #) | (N = 5) |
| Dv 10 | 45.5 |
| Dv 50 | 90.6 |
| Dv 90 | 162 |

TABLE 11
Drug content of Coated Clonidine Resinate (lot 9059-50)

| Sample Name | Coated Clonidine Resinate |
|---|---|
| (Lot #) | (9059-50) |
| Drug content (%) | 3.88 |

### Non-Micronized

### Preparation of Coated Clonidine Resinate (9059-40).

1. Preparation of ion-exchange resin-clonidine resinate (9059-36)
646 mg clonidine HCl was added to 500 ml purified water. Stir until a clear solution is obtained. Weigh 5001 mg Amberlite IRP 64 and add to the clonidine solution. The initial pH of the resulting suspension is 3.19. 0.1N NaOH solution was used to adjust the pH of the suspension to 6.0. Stirring was continued, and the pH was maintained at 6 for 3 hours. The suspension was filtered to collect the resinate particles and dry in an oven at 40° C.
The drug content of the resinate was determined by HPLC and found to be 9.5%.
2. Preparation of Coating Solution.
1602.1 mg Eudragit RS100 was dissolved in 4571 mg Methylene Chloride (CH₂Cl₂) under mechanical mixing until a clear polymer solution is obtained (labeled as Eudragit RS100 Solution 1).
3. Preparation of Resin-Polymer suspension:
2000.7 mg Amberlite IRP64 Resin-Clonidine resinate was dispersed in the above Eudragit RS100 Solution 1 under agitation to form a uniform suspension (labeled as Suspension 1).
4. Prepare a 250 mL 1.5% Polyvinyl Alcohol (PVA) Aqueous Solution (PVA Solution 1);
5. Prepare a 2500 mL 0.5% PVA Aqueous Solution (PVA Solution 2);
6. Add 5 mL Suspension 1 to the PVA Solution 1 while mixing using a homogenizer (PT-MR 3000) for 5 min at 10000 RPM (Dispersion 1).
7. Pass the Dispersion 1 through the Microfluidizer (Microfluidics M-110P) equipped with two microchannel chambers, replacement (front) and 200 μm (back), in serial order with a pressure of 4000 psi.
8. The resulting dispersion is discharged into the PVA Solution 2 under mixing. Continue mixing for 3 hours at room temperature.
9. Collect the coated Clonidine resinate by centrifuging. Wash the collected coated Clonidine resinate twice with purified water. Collect the washed coated Clonidine resinate by centrifuging again.
10. Dry the coated clonidine resinate using a freeze dryer.

The dried coated Clonidine resinate is tested for drug content. The results are provided in Table 12.

TABLE 12
Drug content of Coated Clonidine Resinate (lot 9059-40)

| Sample Name | Coated Clonidine Resinate |
|---|---|
| (Lot #) | (9059-40) |
| Drug content (%) | 5.2 |

The dissolution profiles of the uncoated micronized clonidine resinate, coated non-micronized clonidine resinate, and coated micronized clonidine resinate were tested using the following dissolution method:
Dissolution medium: 500 ml 0.01N HCl solution, 75 rpm, at 37 C, USP apparatus II.

The results are shown in Table 13.

TABLE 13
Dissolution profiles of uncoated and
coated Clonidine Resinate, 0.1 mg

| | Uncoated | Coated Non- | Coated |
|---|---|---|---|
| | Micronized | Micronized | Micronized |
| | Clonidine | Clonidine | Clonidine |
| Time | Resinate | Resinate | Resinate |
| (hours) | (9059-49) | (9059-40) | (9059-50) |
| 0.5 | 97 | 21 | 16 |
| 1 | 98 | 33 | 22 |
| 2 | 98 | 47 | 30 |
| 4 | 98 | 61 | 41 |
| 6 | 99 | 68 | 48 |
| 12 | 98 | 80 | 63 |
| 16 | 99 | 85 | 70 |
| 20 | 99 | 88 | 75 |
| 24 | 99 | 89 | 79 |

The results indicated that without the Eudragit RS100 coating, clonidine was released almost 100% from clonidine resinate in 30 minutes. With the coating, sustained clonidine release can be achieved for both non-micronized and micronized clonidine resinate.

### Example 7

### Preparation of Coated Micronized Viloxazine Resinate by Homogenization Method (8079-10)

In this example, the micronized viloxazine resinate prepared in example 4 is coated using the disclosed coating method with a homogenizer.
1. Preparation of Coating Solution.
5519 mg Eudragit RL100 and 554 mg dibutyl sebacate (DBS) were dissolved in 33316 mg Methylene Chloride (CH₂Cl₂) under mechanical mixing until a clear polymer solution was obtained (labeled as Eudragit RL100 Solution 1).
2. Preparation of Resin-Polymer suspension:
11079 mg viloxazine resinate from example 4 was dispersed in the above Eudragit RL100 Solution 1 under agitation to form a uniform suspension (labeled as Suspension 1).
3. 27 g Polyvinyl Alcohol (PVA) powder was dissolved in 1500 g purified water in a glass bottle with a cap under magnetic stirring. Heat the flask to 87° C. using a hot plate. Mixing was continued until a clear solution was obtained. Cooling down to room temperature. 33.7 g methylene chloride was added to the PVA solution; close the cap and mix for 20 minutes (labeled as PVA Solution).
4. Set up a homogenization system. The system is comprised of a homogenizer equipped with a mixing chamber attached to the rotor head. The rotor can spin at a high speed in the mixing chamber to generate a homogenization effect. The chamber has an inlet and outlet that are fitted with plastic tubing. The outlet plastic tubing was mounted on a peristatic pump which was used to drive the liquid in the plastic tubing. Place both the inlet and outlet tubing ends into the PVA Solution. Turn on the peristatic pump to fill all void space in the chamber and inlet/outlet tubing so that a circulating motion of the PVA Solution can be generated.
5. Measure 24 ml Suspension 1 using a syringe. Turn on the peristatic pump and the homogenizer, inject the Suspension 1 into the PVA Solution via a port on inlet plastic tubing. Continue mixing using the homogenizer (IKA UTL 25 digital Inline ULTRA-TURRAX®) for 11 min at 10000 RPM to obtain the S/O/W dispersion (Dispersion 1).
6. Stop the homogenizer and continue the peristatic pumping to pump all the Dispersion 1 into an open container equipped with a mechanical mixer.
7. Continue mixing for overnight at room temperature.
8. Concentrate the coated viloxazine resinate by centrifuge to remove most of the PVA Solution. Wash the collected coated viloxazine resinate multiple times with purified water to remove the PVA. Collect the washed coated Viloxazine resinate by centrifuging.
9. Dry the coated Viloxazine resinate using a freeze dryer.

The dried coated Viloxazine resinate was tested for drug content. The results are provided in Table 14.

TABLE 14
Drug content of Coated Micronized Viloxazine Resinate

| Sample Name | Coated Viloxazine Resinate |
|---|---|
| (Lot #) | (lot 8079-10) |
| % Viloxazine in coated Resin | 34.71 |
| Measurement 1 | |
| % Viloxazine in coated Resin | 34.81 |
| Measurement 2 | |
| Average (%) | 34.77 |

The dissolution profiles of the coated micronized Viloxazine resinate were tested using the following dissolution method:
Dissolution medium: 900 ml 0.01N HCl solution containing 1% KCl, 75 rpm, at 37 C, USP apparatus II.

The results are shown in Table 15.

TABLE 15
Dissolution profiles of coated Micronized
Viloxazine Resinate, 200 mg

| | Coated Micronized Viloxazine |
|---|---|
| Time (hours) | Resinate (8079-10) |
| 0.5 | 28 |
| 1 | 40 |
| 2 | 54 |
| 4 | 67 |
| 6 | 73 |
| 12 | 77 |
| 16 | 82 |
| 20 | 85 |
| 24 | 87 |

The present disclosure is not to be limited in scope by the specific embodiments described herein. Indeed, various modifications of the invention, in addition to those described herein, will become apparent to those skilled in the art from the foregoing description and the accompanying figures. Such modifications are intended to fall within the scope of the appended claims.

What claimed is:

1. A method of coating ion-exchange resin particles, comprising:
dissolving a coating polymer in a solvent to form a first solution;
suspending ion-exchange resin particles uniformly in the first solution to obtain a solid-in-oil suspension;
adding the solid-in-oil suspension to a second solution that contains a surfactant and is immiscible with the first solution to obtain a solid-in-oil-in-water suspension;
mixing the solid-in-oil-in-water suspension using agitation to form a solid-in-oil-in-water emulsion/suspension (S/O/W dispersion);
passing the S/O/W dispersion through a mechanism or device that exerts a pressure and/or shear force on the S/O/W dispersion and sending resulting dispersion into a third solution that contains a surfactant to obtain a micronized S/O/W dispersion;
removing the solvent from the micronized S/O/W dispersion to deposit and harden the coating polymer on the ion-exchange resin particles to obtain coated ion-exchange resin particles; and
collecting and optionally drying the coated ion-exchange resin particles.

2. The method of claim 1, wherein the ion-exchange resin particles comprise drug complexed resin particles.

3. The method of claim 1, wherein the ion-exchange resin particles comprise micronized drug complexed resin particles having a 90th percentile particle size (D90) less than 50 microns.

4. The method of claim 1, wherein the ion-exchange resin particles comprise cationic or anionic ion-exchange resins.

5. The method of claim 1, wherein the coating polymer forms a barrier on an ion-exchange resin or drug complexed ion-exchange resin particles.

6. The method of claim 1, wherein the coating polymer is insoluble but permeable to a drug in gastrointestinal tract fluid.

7. The method of claim 6, wherein the coating polymer comprises a poly(meth)acrylate polymer, a cellulose derived polymer, a polyvinyl acetate polymer, or a combination thereof.

8. The method of claim 7, wherein the coating polymer is an ethyl acrylate/methyl methacrylate/trimethylammonioethyl mathacrylate copolymer, an ethyl acrylate/methyl methacrylate copolymer, a butyl/methyl methacrylate/dimthylaminoethyl methacrylate copolymer, a methacrylic acid/ethyl acrylate copolymer, a methacrylic acid/methyl methacrylate copolymer, or a combination thereof.

9. The method of claim 1, wherein a concentration of the coating polymer in the first solution is 1-30% wt.

10. The method of claim 1, wherein the solvent is an organic solvent that dissolves the coating polymer while having limited (or less than 10%) solubility in water.

11. The method of claim 1, wherein the solvent comprises methylene chloride, ethyl acetate, chloroform, 1,2-dichloroethane, or a combination thereof.

12. The method of claim 1, wherein the second solution is an aqueous solution containing a surfactant.

13. The method of claim 1, wherein a volume ratio of the first solution to the second solution is from 1:1 to 1:1000.

14. The method of claim 1, wherein the surfactant comprises an ionic surfactant, a non-ionic surfactant, a polymeric surfactant, or a combination thereof.

15. The method of claim 14, wherein the surfactant comprises polyvinyl alcohol.

16. The method of claim 1, wherein a concentration of the surfactant in the second solution is from 0.1% to 10% by volume or by weight.

17. The method of claim 1, wherein the agitation is produced by a high shear mechanical process.

18. The method of claim 17, wherein the high shear mechanical process is homogenization.

19. The method of claim 18, wherein the agitation is produced by a homogenizer operating at a speed of from 500 to 20,000 rpm.

20. The method of claim 1, wherein the mechanism/device has an opening or orifice through which a liquid dispersion can be forced under a pressure of from 100 to 50,000 psi.

21. The method of claim 1, wherein the mechanism or device comprises an in-line homogenizer operating at a speed of from 500 to 20,000 rpm.

22. The method of claim 1, wherein the third solution is an aqueous solution containing a surfactant.

23. The method of claim 1, wherein a volume ratio of the second solution to the third solution is from 1:0 to 1:1000.

24. The method of claim 1, wherein the surfactant in the third solution is an ionic surfactant, a non-ionic surfactant, a polymeric surfactant, or a combination thereof.

25. The method of claim 24, wherein the surfactant in the third solution comprises polyvinyl alcohol.

26. The method of claim 1, wherein a concentration of the surfactant in the second solution is from 0.01% to 5% by volume or by weight.

27. The method of claim 1, wherein the solvent is removed by heating and/or vacuum.

28. The method of claim 27, wherein the solvent is removed by heating the third solution to a temperature of from 25 to 90° C.

29. The method of claim 1, wherein the coated ion-exchange resin particles are collected by filtration, centrifuging, or centrifuge filtration.

30. The method of claim 1, wherein the coated ion-exchange resin particles are dried by heating or freeze-drying.

31. The method of claim 1, wherein a weight ratio of the coating polymer to the ion-exchange resin particles is from 1% to 80%.

32. The method of claim 1, wherein the ion-exchange resin particles have a particle size of from 0.5 to 500 microns.

33. The method of claim 32, wherein the ion-exchange resin particles have a particle size of from 0.5 to 50 microns.

34. The method of claim 1, wherein the S/O/W dispersion has a droplet size range of from 1 to 1000 microns.

35. The method of claim 34, wherein the micronized S/O/W dispersion has a droplet size range of from 0.5 to 50 microns.

36. The method of claim 1, wherein the coated ion-exchange resin particles have a particle size of D90 smaller than 50 microns.
